(19) United States
(12) Patent Application Publication
(10) Pub. No.: US 2022/0280765 A1
(43) Pub. Date: Sep. 8, 2022

(54) Shockwave-Enhanced Drug-Delivery Catheter

(71) Applicant: Spectrumedics Medical Technology (Shanghai) Co., Ltd., Shanghai (CN)

(72) Inventors: Al Ray Tubio Tabiliran, Singapore (SG); Hui Qun Phang, Bishan View (SG); Xian Wei Jeremy Goh, Singapore (SG)

(21) Appl. No.: 17/633,493

(22) PCT Filed: Aug. 6, 2020
(86) PCT No.: PCT/SG2020/050459

§ 371 Date: Feb. 7, 2022

Related U.S. Application Data
(60) Provisional application No. 62/884,281, filed on Aug. 8, 2019.

(51) Int. Cl. A61M 37/00; A61M 25/10

(52) U.S. Cl. CPC A61M 37/0092; A61M 25/1006; A61M 25/1011; A61M 2025/105; A61M 2025/1052; A61M 2025/1086

(57) ABSTRACT

A drug delivery catheter includes a first axially extending elongate member, a second axially extending elongate member, and a main chamber wall. The inner surface of the main chamber wall defines a main chamber. The distal end of the main chamber wall is joined to the distal end of the second axially extending elongate member and the proximal end of the main chamber wall is joined to the distal end of the first axially extending elongate member such that the main chamber is disposed over a distal portion of at least the second elongate member. One or more shockwave-generating elements are disposed within the main chamber. The main chamber is in fluid communication with the lumen of the first elongate member. The drug delivery catheter is configured to deliver an active agent to a target site through the lumen of the first elongate member.

### FIELD OF INVENTION

The present invention relates to the field of medical devices, and in particular to a drug-delivery catheter that utilizes shockwaves to enhance drug delivery. The device of the invention may be useful in the treatment of diseased vessels within a body, particularly the treatment of atherosclerosis associated with coronary artery disease (CAD) and peripheral artery disease (PAD).

### BACKGROUND OF THE INVENTION

Localized drug delivery within the vasculature is the act of treating a specific bodily vessel with therapeutic agents without utilizing the circulatory system of the body. This allows the drug to interact only with the desired regions, increasing the effectiveness of the treatment, and avoiding possible damage to healthy, non-targeted tissue. In recent times, there has been rapid development in the use of drug-infused balloon (DIB) catheters as a means of delivering anti-proliferative drugs to an atherosclerotic vessel re-opened by balloon angioplasty.

Coronary artery disease (CAD) and peripheral artery disease (PAD) are obstructions in the arteries of the heart and peripherals caused by the buildup of calcified plaque. An emerging method of treatment is the use of drug-coated balloon (DCB) angioplasty. After navigating a standard balloon angioplasty catheter to the stenosed artery, the water-tight balloon is inflated with a liquid to re-open the artery, improving blood flow. The surface-coated drug, often an anti-proliferative such as paclitaxel, is forced by the compression into the vessel walls to prevent restenosis of the vessel. Drug infusion during balloon angioplasty has a proven effectiveness in reducing restenosis with rates similar to that of drug-eluting stents (DES), with the added benefit of having no permanent implants placed within the vasculature. Where stent breakage is prone to occurring, such as in the femoropopliteal arteries of the leg, DCBs present an advantage in long-term sustainability of the initial angioplasty treatment.

Problems with DCBs however, limit their real-world effectiveness. For example, having the drug coated directly on to the outer surface of the balloon, even chemically, poses a number of challenges. The drug may begin to immediately wash and slough off the balloon surface once the catheter enters the bloodstream, thus necessitating a relatively large initial amount of drug applied to the balloon surface so that there may be enough remaining once the target site is reached. If a protective sheath is used, the profile of the catheter would have to be increased, which is undesirable for navigating smaller vessels such as those of the heart. Also, forcing the drug into the vessels by pure compression may not always provide the best uptake.

Shockwaves and other vibrations have been used to break up plaques that form on the inside of blood vessels such as arteries. Typically, these treatments involve the use of ultrasound, radiofrequency radiation and/or lasers. All of these methods have the drawback that they produce significant amounts of heat and carry a risk of thermal injury. The generation of ultrasound causes large amounts of heat as a by-product. Generation of laser beams and radiofrequency waves also produces significant amounts of heat, and these forms of energy are also prone to causing heat injuries due to their ability to rapidly heat bodily tissues. The risks of these types of catheter are discussed in the art as set out below.

Owens, Seminars in Interventional Radiology, vol. 25, no. 1, 2008, pp. 37-41 discusses the ultrasound-based EKOS EndoWave Infusion Catheter System, and notes that the EKOS system requires normal saline to be infused via a coolant port connected to a central lumen and used to diffuse heat energy generated by the ultrasound transducer. More generally, a skilled person would be well aware that ultrasound transducers produce large amounts of heat and must be cooled.

Haines and Watson, PACE, vol. 12(6), 1989, pp. 962-976, and Morosawa et al., Journal of the American Heart Association, 8, 2019, pp. 1-13, both discuss the issue of thermal injury arising from radiofrequency ablation treatment, and Morosawa notes that such thermal injury is inevitable during radiofrequency catheter ablation.

Laser ablation treatment can also cause thermal damage to the vessel walls. When lasers are instead used to generate shockwaves in aqueous fluid, this does involve overheating risks. In this regard, Morosawa notes that lasers cannot be pulsed continuously or they would overheat, and such laser-based shockwave catheters include a water supply and drainage system to remove surplus heat in the water in the shockwave reflector (page 2, left column, “SWCA system”).

In contrast, shockwaves can be applied to deep lesions without heating the tissue. As such, if shockwaves can be generated in a way that does not produce excess heat, an effective angioplasty treatment may be provided. In addition, the use of shockwaves allows for the exploitation of the cavitation effect by collapsing microbubbles that create powerful cavitation jets, further enhancing the penetration power of drugs through the vessel walls

### BRIEF SUMMARY OF THE INVENTION

The problems associated with the prior art are overcome by the present invention, which uses methods such as electrical arc discharge to produce shockwaves without significant heat as a by-product.

Accordingly, the present invention provides the following numbered clauses.

1. A drug delivery catheter comprising:
a first axially extending elongate member comprising a lumen, an outer surface, a distal end and a proximal end;
a second axially extending elongate member comprising an outer surface, a distal end and a proximal end;
a main chamber wall having an outer and inner surface, a distal end, and a proximal end, the inner surface of the main chamber wall defining a main chamber, where the distal end of the main chamber wall is joined to the distal end of the second axially extending elongate member and the proximal end of the main chamber wall is joined to the distal end of the first axially extending elongate member such that the main chamber is disposed over a distal portion of at least the second elongate member; and
one or more shockwave-generating elements disposed within the main chamber, wherein:
the main chamber is in fluid communication with the lumen of the first elongate member;
the drug delivery catheter is configured to deliver an active agent to a target site through the lumen of the first elongate member; and
the one or more shockwave-generating elements comprise two or more electrodes disposed on the outer surface of the second elongate member, the electrodes being configured to enable the arcing of an electrical discharge between electrodes when a potential difference is applied, thereby giving rise to shockwaves.

2. The drug delivery catheter of Clause 1, further comprising one or more chamber walls, each defining a further chamber, wherein each of the further chamber(s) is/are fluidly connected to a lumen of an elongate member and/or to a fluid channel in a lumen of an elongate member, such that each of the further chamber(s) is/are fluidly isolated from the main chamber.

3. The drug delivery catheter of Clause 2, wherein a first of the one or more further chambers is disposed over a portion of the second elongate member within the main chamber and the one or more shockwave-generating elements are disposed within the first of the one or more further chambers.

4. The drug delivery catheter of Clause 2 or Clause 3, further comprising a third axially extending elongate member having an outer surface, a distal end, a proximal end and a lumen, wherein the first of the one or more further chambers is in fluid communication with the lumen of the third elongate member.

5. The drug delivery catheter of Clause 4, wherein:

the third axially extending elongate member is partly housed within the lumen of the first elongate member, and extends from the distal end of the first elongate member; and

the second axially extending elongate member is partly housed within the lumen of the third elongate member, and extends from the distal end of the third elongate member.

6. The drug delivery catheter of Clause 4 or Clause 5, wherein the proximal end of the first of the one or more further chambers is attached to the distal end of the third elongate member, and the distal end of the first of the one or more further chambers is attached to the second elongate member.

7. The drug delivery catheter of any one of the preceding clauses, wherein the two or more electrodes comprise one or more electrode pairs disposed on the outer surface of the second elongate member, the electrode pairs being configured to enable the arcing of an electrical discharge between electrodes when a potential difference is applied, thereby giving rise to shockwaves.

8. The drug delivery catheter of any one of the preceding clauses, wherein the wall of the main chamber comprises a plurality of micropores extending through the wall to fluidly connect the interior of the main chamber to the exterior of the main chamber, optionally wherein the micropores have a diameter of from 1 to 2 μm.

9. The drug delivery catheter of any one of Clauses 2 to 7, wherein:

the one or more further chambers comprise one or more fluid pockets having a distal end, a proximal end, and an outer surface, each fluid pocket running along the length of the interior surface of the main chamber wall or along at least part of the length of the exterior surface of the main chamber wall;

each fluid pocket is enclosed at its distal end; and the proximal end of the fluid pocket is in fluid communication with a lumen of an elongate member and/or to a fluid channel in a lumen of an elongate member.

10. The drug delivery catheter of Clause 9, wherein the one or more fluid pockets are a plurality of fluid pockets.

11. The drug delivery catheter of Clause 10, wherein each fluid pocket is in fluid communication with a fluid channel within the lumen of the first elongate member, provided that each fluid channel is fluidly isolated from the main chamber.

12. The drug delivery catheter of any one of Clauses 9 to 11, wherein when a fluid pocket is arranged to run along at least part of the length of the exterior surface of the main chamber wall, the outer surface of each fluid pocket comprises a plurality of micropores extending through the surface of the fluid pocket to fluidly connect the inner volume of fluid pocket to the exterior of the main chamber, optionally wherein the micropores have a diameter of from 1 to 2 μm.

13. The drug delivery catheter of any one of Clauses 9 to 11, wherein when a fluid pocket is arranged to run along the interior surface of the main chamber wall, the wall of the main chamber comprises a plurality of micropores extending through the wall to fluidly connect the interior of the fluid pocket to the exterior of the main chamber, optionally wherein the micropores have a diameter of from 0.1 to 5 μm, optionally from 1 to 2 μm.

14. The drug delivery catheter of any one of the preceding clauses, further comprising:

a proximal occluding element disposed over a region of the outer surface of an elongate member comprising a lumen, where said proximal occluding element is more proximal than the main chamber; and

a distal occluding element disposed over a region of the outer surface of an elongate member comprising a lumen, where said distal occluding element is more distal than the main chamber,

wherein the proximal and distal occluding elements are each configured to reversibly occlude a vessel, thereby isolating a portion of said vessel between the proximal and distal occluding elements.

15. The drug delivery catheter of Clause 14, wherein:

the proximal occluding element has an inner surface defining an inflatable proximal occluding chamber; and

the distal occluding element has an inner surface defining an inflatable distal occluding chamber.

16. The drug delivery catheter of Clause 15, wherein:

the proximal occluding element is inflatable via an opening on the region of the outer surface of the elongate member encompassed by the proximal occluding element, the opening connecting the proximal occluding chamber to the lumen of the elongate member encompassed by the proximal occluding element and/or to a fluid channel within the lumen of the elongate member encompassed by the proximal occluding element;

the distal occluding element is inflatable via an opening on the region of the outer surface of the elongate member encompassed by the distal occluding element, the opening connecting the distal occluding chamber to the lumen of the elongate member encompassed by the distal occluding element and/or to a fluid channel within the lumen of the elongate member encompassed by the distal occluding element.

17. The drug delivery catheter of Clause 16, wherein the proximal occluding element is disposed over the outer surface of the first elongate member proximal to the main chamber;

the distal occluding element is disposed over the outer surface of a distal portion of the second elongate member;

and the second elongate member has a lumen.

18. The drug delivery catheter of any one of Clauses 14 to 17, further comprising an outer axially extending elongate member having a distal end, a proximal end, an outer surface and a lumen, where the first axially extending elongate member is partly housed within the lumen of the outer elongate member and extends from the distal end of the outer elongate member; and the proximal occluding element is disposed on the outer surface of the outer elongate member.

19. The drug delivery catheter of any one of Clauses 14 to 18, wherein an elongate member comprises one or more openings along its length, the one or more openings being positioned between the proximal and distal occluding elements, each opening connecting a fluidly separate fluid channel within the lumen of said elongate member to the exterior of the catheter, optionally wherein the elongate member comprising one or more openings is the first elongate member.

20. The drug delivery catheter of any one of Clauses 14 to 19, wherein the proximal and distal occluding elements are balloons.

### BRIEF DESCRIPTION OF THE DRAWINGS

FIG. 1 shows a basic shockwave-generating balloon catheter.

FIG. 2 shows a first type of drug delivery catheter system using a single microporous balloon.

FIG. 3A shows a second type of drug delivery catheter system; a variation of the system in FIG. 2 where the balloon comprises a series of fluid pockets attached to the balloon wall.

FIG. 3B shows a side view of the fluid pocket-laden single balloon in FIG. 3A.

FIG. 3C shows a sectioned view of the fluid pocket-laden single balloon in FIG. 3A, exposing the proximal connection of each fluid pocket with fluid channels within the first elongate member.

FIG. 3D shows a cross-sectioned view of fluid channels within the lumen of first axially extending elongate member, showing where the proximal ends of each fluid pocket of FIG. 3C connect to.

FIG. 4 shows another configuration of the fluid pocket-comprising single balloon in FIG. 3A-C, where the fluid pockets do not run parallel to the axis of the catheter, but rather traverse at least some extent of the circumference as well.

FIG. 5A shows a depiction of a third type of drug delivery catheter system using a double-balloon configuration.

FIG. 5B shows a sectioned view of the double-balloon configuration shown in FIG. 5A showing the separation between balloons and the flow of fluids.

FIG. 6 shows a fourth type of drug delivery catheter system using a multiple-balloon setup. The catheter comprises proximal and distal occluding balloons in addition to the single main balloon shown in FIG. 1.

FIG. 7A shows an alternative double-balloon configuration where the elongate members are adjacent to each other.

FIG. 7B shows a sectioned view the alternative type of double-balloon configuration shown in FIG. 7A.

FIG. 8 shows the stepwise method of usage for the catheter of FIG. 2.

FIG. 9 shows the stepwise method of usage for the catheter of FIG. 3A-D.

FIG. 10 shows the stepwise method of usage for the catheter of FIG. 5A-B.

FIG. 11 shows the stepwise method of usage for the catheter of FIG. 6.

### DETAILED DESCRIPTION

Described herein are drug delivery catheter systems that utilize chambers containing shockwave emitters for the purpose of enhancing the uptake of therapeutic medicinal agents by bodily vessels. The devices and their use cases relate mainly to the treatment of coronary artery disease (CAD) and peripheral artery disease (PAD), but should be understood to enable shockwave-enhanced delivery of drugs of any type within any bodily vessel where a catheter may be tracked.

In the present invention, a reference to the lumen of an axially extending elongate member includes a reference to the lumen of any elongate member that is within the lumen of the aforesaid axially extending elongate member.

A basic single-chamber catheter drug delivery system may comprise an axially extending first elongate member with an outer surface, and a central lumen comprising one or more fluid channels through which fluid may flow. The central lumen of the first elongate member may partly house a second axially extending elongate member. A plurality of shockwave-generating elements may be disposed over the outer surface of the second elongate member. Each shockwave-generating element may constitute a two or more electrodes (e.g. a pair of electrodes or a set of 3 or more electrodes, as discussed below) that are configured to provide electrical arc discharge when a potential difference is applied between the electrodes. In use, the most proximal electrode (e.g. of the most proximal electrode pair) may be connected to a first electrically-conductive wire that electrically joins said electrode to a first output terminal of a high voltage source. The most distal electrode (e.g. of the most distal electrode pair) may be connected to a second electrically-conductive wire that electrically joins said electrode to a second output terminal of the high voltage source. The first and second wires may run over the outer surface of the first elongate member, and may be sealably bonded to this outer surface by an adhesive, or crimped down by insulating jacket. This configuration results in a series circuit that joins all the electrodes to the high voltage source and, in the presence of a fluid medium, allows the generation of the electrical arc discharge between physically-disconnected electrode pairs to produce shockwaves. In some instances, two or more series circuits may be formed, by increasing the number of wired electrodes and increasing the number of connected output terminals on the high voltage source. This may be desirable when a larger number of electrodes are used. Thus, when more than 4 electrode pairs (8 electrodes) are used, they may be arranged in two or more series circuits, which, reduces the voltage requirement for each circuit as compared to arranging the electrodes in a single circuit.

The second elongate member may have within it, a central lumen that slidably receives a wire guide of appropriate size according to one intended application of the device. An inflatable chamber may encompass the length of the second elongate member containing the shockwave sources, which may have a medial length section that expands to a fixed diameter upon filling with a fluid. The proximal end of the chamber may be of a non-expandable diameter and may be affixed to the outer surface at the distal end of the first elongate member. The distal end of the chamber, similarly, may be of a non-expandable diameter and may be affixed to the outer surface of the distal region of the second elongate member. The chamber may thus sealably enclose the shockwave sources within its inner volume, and may be inflated by a fluid flushed through a fluid channel within the lumen of the first elongate member from the proximal end. Shockwaves produced by the shockwave sources within the chamber propagate through the liquid and pass through the boundary of the chamber to the contacting media outside.

Treatment using the catheter of the invention typically comprises: inserting the catheter into a bodily vessel through conventional means and tracking it to an occlusion, inflating the chamber with a fluid until the chamber impinges upon and makes full contact with the target lesion, applying a potential difference across the circuit to produce shockwaves to crack calculi in the lesion, and deflating the chamber and withdrawing the catheter once complete.

The shockwaves produced by catheters according to the invention can serve a dual purpose. First, the shockwaves can be used to fracture plaques formed on the interior of a vessel. Second, by varying the energy intensity and number of pulses delivered to tissue, the shockwaves can help stimulate the uptake of drugs released by the catheter. The intended purpose of the shockwaves will depend on the nature of the lesion(s) to be treated. Non-calcified lesions do not need to be fractured, and so the treatment procedure will utilise shockwaves to enhance drug uptake and penetration into the tissue. For treatment of calcified lesions, the procedure will involve two steps: A first shockwave calcium fracturing step which typically does not involve drug infusion/release from the catheter; and a second drug penetration enhancement step in which drug is released/infused from the catheter along with simultaneous and/or sequential shockwave production to enhance the update/penetration of the drug.

The catheters of the invention comprise a main chamber. The main chamber may preferably be an inflatable chamber, such as a balloon. Where other chambers are present, they may also be inflatable chambers such as balloons. A reference hereinafter to a balloon includes a reference to a (inflatable) chamber.

In one embodiment of the single-chamber device, the exterior wall of the chamber may have a plurality of holes disposed over its surface, enabling the inner volume of the chamber to be in fluid communication with the volume of media on the outside of the chamber. The holes may have a diameter that is small enough to allow the main chamber to be inflated, but large enough for fluid within the main chamber to escape out of the main chamber once the main chamber is inflated, particularly upon the application of elevated pressure. Suitable hole sizes are typically on the scale of micrometers in diameter, and the holes may typically have a diameter in the range of about 0.1 to about 5 μm, such as a diameter of from about 1 to about 2 μm. The holes may span the entire medial length of the chamber. This embodiment is used in the same way as a basic shockwave angioplasty catheter. However, instead of simply inflating the chamber with a fluid, a drug solution may be flushed through a fluid channel within the lumen of the first elongate member in order to inflate the chamber. Upon full inflation, added pressure may cause the drug to seep out of the pores of the chamber through to the outside media at high velocity. It is at this point that the shockwave sources may be triggered, generating shockwaves that accelerate the uptake of the drug into the vessel walls.

In another embodiment of the single-chamber device, the exterior wall of the chamber may have disposed upon its outer, or preferably inner surface, a plurality of fluid pockets running along the length of the chamber from its proximal end, for example until the distal end of the medial length of the chamber. Each fluid pocket may have a proximal end that terminates at the proximal end of the chamber. Each proximal end may be in fluid communication with a fluid channel which may be within the lumen of the first elongate member. The fluid channel may be disposed over the inner or outer surface of the first elongate member. Alternatively, the first elongate member could house a further elongate member between the first and second elongate members, creating an annular fluid channel between the first and further elongate members. The fluid pockets may be in fluid connection with such an annular fluid channel.

The fluid pockets are fluidly isolated from the main chamber (i.e. are not in fluid communication with the main chamber). The fluid pockets may be filled with a fluid through a fluid channel, for example a fluid channel within the lumen of the first elongate member. Located on the outer surface of each fluid pocket may be a plurality of holes (i.e. micropores) that, similar to the previous embodiment, enable the inner volume of each fluid pocket to be in fluid communication with the volume of media on the outside of the main chamber. The holes may have a similar diameter range of 1-2 μm.

When the fluid pockets are present on the inner surface of the wall of the chamber, the outer surface of the fluid pocket will be the outer surface of the wall of the main chamber, and the micropores will be present on the portion of the wall of the main chamber that forms the outer surface of the fluid pocket. When the fluid pocket is present on the outer surface of the wall of the main chamber, the outer surface of the fluid pocket will be separate to the outer surface of the wall of the chamber, and the micropores will be present on the outer surface of the fluid pocket. In this embodiment, the micropores are preferably only present on the outer surface of the fluid pockets (i.e. preferably no micropores connect the main chamber to the exterior of the main chamber). For this embodiment, after tracking the catheter to the lesion site, the main chamber may first be inflated with a fluid, thereafter the drug solution may be infused via a fluid channel, filling the plurality of fluid pockets. Upon full inflation, added pressure may be applied to either the main chamber or to each individual fluid pocket in order to increase the rate of drug delivery. Shockwaves may also be applied at this point to aid in drug uptake by the vessel walls.

The fluid pockets running along the length of the chamber may span as well, at least some part of the circumference of the chamber. This may result for example, in a helical path across the chamber's surface, for the purpose of covering a larger surface area of vessel that receives the medication. Multiple fluid pockets may form multiple helices to further increase the density of pores present on the surface of the main chamber.

A variation of the single-chamber catheter drug delivery system utilizes a second chamber in addition to the main chamber encompassing the shockwave sources. A basic double-chamber catheter drug delivery system may comprise the entirety of the basic single-chamber system including any porous modifications to the main chamber. A double-chamber system may comprise a third axially extending elongate member with a central lumen comprising one or more fluid channels, the third elongate member partly housed by the first elongate member, and the third elongate member partly houses the second elongate member. The third elongate member has a distal end that is attached to the proximal end of the second chamber, the distal end of the second chamber being attached to the second elongate member. The main chamber forms an outer chamber which may encompass the second chamber in its entirety. The inner (second) chamber encompasses the shockwave source(s), and may be inflated with a fluid to expand to a pre-determined diameter smaller than that for the main chamber. The proximal and distal ends of the second chamber are similarly non-expandable. The proximal end may be affixed to the outer surface of the distal end of the third elongate member, while the distal end may be affixed to the outer surface of the distal region of the second elongate member.

As for the single chamber device, in one embodiment of the double-chamber device, the outer (first) chamber may have a plurality of holes disposed over its surface, enabling the inner volume of the outer chamber to be in fluid communication with the volume of media on the outside of the chamber. The holes on the chamber may be on the scale of micrometers in diameter, ranging from 1-2 μm, and may span the entire medial length of the chamber. The inner (second) chamber may first be inflated upon entering the treatment area of the vessel. After full inflation, the outer (first) chamber may then be inflated with the drug solution via the fluid channel to which it is connected. Added pressure may then be applied to either the inner or outer chamber in order to increase the rate of delivery of the drug through the pores of the outer chamber. Shockwaves may be applied at this point to aid in the rate of drug uptake by the vessel walls.

Another variation of the (typically single-chambered) catheter drug delivery system utilizes two occluding elements, located proximal and distal to the main chamber. The purpose of these occluding elements is to anchor the catheter within a section of the vessel, and occlude it. The cavity created by the occluding elements can then be evacuated of blood (e.g. by evacuation through an opening in one of the elongate members, such as the first elongate member), while the main chamber is inflated to a set diameter smaller than the internal diameter of the vessel. Drug infusion may take place at this point in order to fill the cavity with the drug (e.g. infusion through a chamber or fluid pocket as described in an embodiment above, or through an opening present on a portion of one of the elongate members which is in contact with the cavity between the two occluding elements). In this embodiment, the lumen of one of the elongate members could comprise multiple fluid channels, such that the aforementioned cavity in the vessel can be evacuated through one opening in the respective elongate member via one fluid channel, and active drugs can be delivered to the cavity through another opening via a different fluid channel. The proximal and/or distal occluding elements may be inflatable and be inflated via separate partitions in this, or another, elongate member. Additional pressure may be applied in order to force the drug into the vessel walls. Shockwaves may also be applied in order to aid in the rate of drug uptake by the vessel walls.

The occluding elements are preferably inflatable occluding elements, for example balloons. The occluding elements may each have an inner surface defining an occluding chamber which is in fluid communication with a fluid channel within the lumen of the elongate member over which the occluding element is disposed. In this way, fluid may be pumped into the occluding elements to inflate them and occlude a vessel.

In this embodiment, the main chamber may be fluidly isolated from the cavity formed by the occluding elements. Therefore, the fluid within the main chamber which is exposed to the shockwave source does not come into contact with the tissue of a patient.

A person skilled in the art will understand that the source of shockwaves used in the present invention is not particularly limited, provided that the source of shockwaves does not produce large amounts of heat. In general, shockwaves may be created by the cavitation bubble effect. The source of shockwaves is preferably electrical arc discharge, i.e. shockwaves produced by a series of two or more electrodes, optionally arranged in pairs, configured to produce electrical arc discharge when a potential difference is applied across the electrodes. This is preferable because the use of electrodes (e.g. pairs of electrodes) to generate electrical arc discharge does not produce large amounts of heat. Ultrasound, and other methods such as lasers and radiofrequency radiation, produces large amounts of heat that must be carefully dissipated in order to avoid damaging the shockwave source and/or causing a thermal injury to a patient.

A person skilled in the art would understand that the number of shockwave sources is not particularly limited. The catheter could comprise a single shockwave source, two shockwave sources, three shockwave sources, four shockwave sources or more than four shockwave sources. When the shockwave source comprises two or more electrodes, it may comprise additional electrodes (optionally arranged in pairs) to increase the number of points at which electrical arc discharge may be generated. For example, the shockwave source may comprise from 2 to 24 electrodes, such as from 2 to 20 electrodes, from 4 to 16 electrodes, from 4 to 12 electrodes or from 4 to 8 electrodes, any of which may be arranged in pairs. While a pair of electrodes provides one gap for electrical arc discharge, three electrodes can be used to provide two gaps for electrical arc discharge, potentially increasing the intensity of shockwaves. Thus, the catheter may comprise pairs of electrodes, such as 2, 4, 6, 8, 10, 12, 14, 16, 18, 20, 22 or 24 electrodes (e.g. 2, 4, 6, 8 or 10 electrodes for a smaller catheter, or 12, 14, 16, 18, 20, 22 or 24 electrodes if a larger catheter is desired). Alternatively, the catheter may comprise an odd number of electrodes, such as 3, 5, 7, 9, 11, 13, 15, 17, 19, 21 or 23 electrodes (e.g. 3, 5, 7, or 9 electrodes for a smaller catheter, or 11, 13, 15, 17, 19, 21 or 23 electrodes for a larger catheter). As will be appreciated by a person skilled in the art, a catheter comprising an even number of electrodes need not only comprise electrode pairs. For example, a catheter comprising 6 electrodes may comprise two sets of three electrodes.

For the avoidance of doubt, it is explicitly contemplated that where a number of numerical ranges related to the same feature are cited herein, that the end points for each range are intended to be combined in any order to provide further contemplated (and implicitly disclosed) ranges. Thus, in relation to the number of electrodes present in the catheter, there is disclosed:

2 to 24, 2 to 23, 2 to 22, 2 to 21, 2 to 20, 2 to 19, 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4, and 2 to 3 electrodes;

3 to 24, 3 to 23, 3 to 22, 3 to 21, 3 to 20, 3 to 19, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, and 3 to 4 electrodes;

4 to 24, 4 to 23, 4 to 22, 4 to 21, 4 to 20, 4 to 19, 4 to 18, 4 to 17, 4 to 16, 4 to 15, 4 to 14, 4 to 13, 4 to 12, 4 to 11, 4 to 10, 4 to 9, 4 to 8, 4 to 7, 4 to 6, and 4 to 5 electrodes;

5 to 24, 5 to 23, 5 to 22, 5 to 21, 5 to 20, 5 to 19, 5 to 18, 5 to 17, 5 to 16, 5 to 15, 5 to 14, 5 to 13, 5 to 12, 5 to 11, 5 to 10, 5 to 9, 5 to 8, 5 to 7, and 5 to 6 electrodes;

6 to 24, 6 to 23, 6 to 22, 6 to 21, 6 to 20, 6 to 19, 6 to 18, 6 to 17, 6 to 16, 6 to 15, 6 to 14, 6 to 13, 6 to 12, 6 to 11, 6 to 10, 6 to 9, 6 to 8, and 6 to 7 electrodes;

7 to 24, 7 to 23, 7 to 22, 7 to 21, 7 to 20, 7 to 19, 7 to 18, 7 to 17, 7 to 16, 7 to 15, 7 to 14, 7 to 13, 7 to 12, 7 to 11, 7 to 10, 7 to 9, and 7 to 8 electrodes;

8 to 24, 8 to 23, 8 to 22, 8 to 21, 8 to 20, 8 to 19, 8 to 18, 8 to 17, 8 to 16, 8 to 15, 8 to 14, 8 to 13, 8 to 12, 8 to 11, 8 to 10, and 8 to 9 electrodes;

9 to 24, 9 to 23, 9 to 22, 9 to 21, 9 to 20, 9 to 19, 9 to 18, 9 to 17, 9 to 16, 9 to 15, 9 to 14, 9 to 13, 9 to 12, 9 to 11, and 9 to 10 electrodes;

10 to 24, 10 to 23, 10 to 22, 10 to 21, 10 to 20, 10 to 19, 10 to 18, 10 to 17, 10 to 16, 10 to 15, 10 to 14, 10 to 13, 10 to 12, and 10 to 11 electrodes;

11 to 24, 11 to 23, 11 to 22, 11 to 21, 11 to 20, 11 to 19, 11 to 18, 11 to 17, 11 to 16,

11 to 15, 11 to 14, 11 to 13, and 11 to 12 electrodes; 12 to 24, 12 to 23, 12 to 22, 12 to 21, 12 to 20, 12 to 19, 12 to 18, 12 to 17, 12 to 16, 12 to 15, 12 to 14, and 12 to 13 electrodes;

13 to 24, 13 to 23, 13 to 22, 13 to 21, 13 to 20, 13 to 19, 13 to 18, 13 to 17, 13 to 16, 13 to 15, and 13 to 14 electrodes;

14 to 24, 14 to 23, 14 to 22, 14 to 21, 14 to 20, 14 to 19, 14 to 18, 14 to 17, 14 to 16, and 14 to 15 electrodes;

15 to 24, 15 to 23, 15 to 22, 15 to 21, 15 to 20, 15 to 19, 15 to 18, 15 to 17, and 15 to 16 electrodes;

16 to 24, 16 to 23, 16 to 22, 16 to 21, 16 to 20, 16 to 19, 16 to 18, and 16 to 17 electrodes;

17 to 24, 17 to 23, 17 to 22, 17 to 21, 17 to 20, 17 to 19, and 17 to 18 electrodes;

18 to 24, 18 to 23, 18 to 22, 18 to 21, 18 to 20, and 18 to 19 electrodes;

19 to 24, 19 to 23, 19 to 22, 19 to 21, and 19 to 20 electrodes;

20 to 24, 20 to 23, 20 to 22, and 20 to 21 electrodes;

21 to 24, 21 to 23, and 21 to 22 electrodes;

22 to 24, and 22 to 23 electrodes; and

23 to 24 electrodes.

In some embodiments of the invention, the electrodes are arranged as electrode pairs. Thus, in relation to the number of electrode pairs present in the catheter, there is disclosed:

1 to 12, 1 to 11, 1 to 10, 1 to 9, 1 to 8, 1 to 7, 1 to 6, 1 to 5, 1 to 4, 1 to 3, and 1 to 2 electrode pairs;

2 to 12, 2 to 11, 2 to 10, 2 to 9, 2 to 8, 2 to 7, 2 to 6, 2 to 5, 2 to 4, and 2 to 3 electrode pairs;

3 to 12, 3 to 11, 3 to 10, 3 to 9, 3 to 8, 3 to 7, 3 to 6, 3 to 5, and 3 to 4 electrode pairs;

4 to 12, 4 to 11, 4 to 10, 4 to 9, 4 to 8, 4 to 7, 4 to 6, and 4 to 5 electrode pairs;

5 to 12, 5 to 11, 5 to 10, 5 to 9, 5 to 8, 5 to 7, and 5 to 6 electrode pairs;

6 to 12, 6 to 11, 6 to 10, 6 to 9, 6 to 8, and 6 to 7 electrode pairs;

7 to 12, 7 to 11, 7 to 10, 7 to 9, and 7 to 8 electrode pairs;

8 to 12, 8 to 11, 8 to 10, and 8 to 9 electrode pairs;

9 to 12, 9 to 11, and 9 to 10 electrode pairs;

10 to 12, and 10 to 11 electrode pairs; and

11 to 12 electrode pairs.

Four main classes of drug delivery catheters are disclosed hereinbelow, each with their own unique features to enhance the efficacy of drug uptake by bodily vessels. The first comprises a single microporous chamber system that applies shockwave energy directly to a drug infusion medium. The second comprises a single microporous chamber system with multiple intra-chamber fluid pockets to carry the drug. The third comprises a double-chamber system where drugs are infused in a microporous outer chamber, and where the inner chamber contains the shockwave emitters. The last comprises a system that occludes and isolates a length of vessel to be perfused with the drug and stimulated with shockwaves.

The drug delivery catheters described herein may comprise micropores or micro-perforations in the first and/or further chambers. The micropores allow the passage of fluids from the respective chamber to the exterior of that chamber. If the interior of the chamber comprises a solution containing a drug, that drug can pass through the micropores to the exterior of the chamber, and be absorbed by the vessel walls. The rate of flow of the drug through the micropores can be increased if a pressure gradient is formed across the chamber wall. Such a pressure gradient can be formed by increasing the pressure inside the first and/or further chambers, such as by inflation of the main chamber, inflation of the fluid pockets, inflation of a further chamber and/or the generation of shockwaves inside the first or a further chamber.

The drug delivery catheters of the present invention comprise two or more axially extending elongate members. These members may be arranged in multiple ways. For example, the elongate members may be arranged within each other in a telescopic fashion, i.e. the first elongate member may partly house the second elongate member, and other elongate members may be present either between the first and second members, or outside the first member. Alternatively, the elongate members may be arranged adjacent to each other. When the elongate members are adjacent to each other they may be attached together along their entire lengths, or may be unattached for all or part (e.g. most) of their lengths. When substantially the entire length of the elongate members is unattached, they may nonetheless be attached at their distal ends where the chamber(s) encompass one or more of the elongate members. While in certain embodiments described herein the axially extending elongate members are described as being housed within other axially extending elongate members, a person skilled in the art would understand that they may alternatively be placed adjacent to each other in a side-by-side fashion. A person skilled in the art would be able to make such a modification to the devices disclosed herein.

Specific embodiments of the invention are described in more detail below with reference to the Figures.

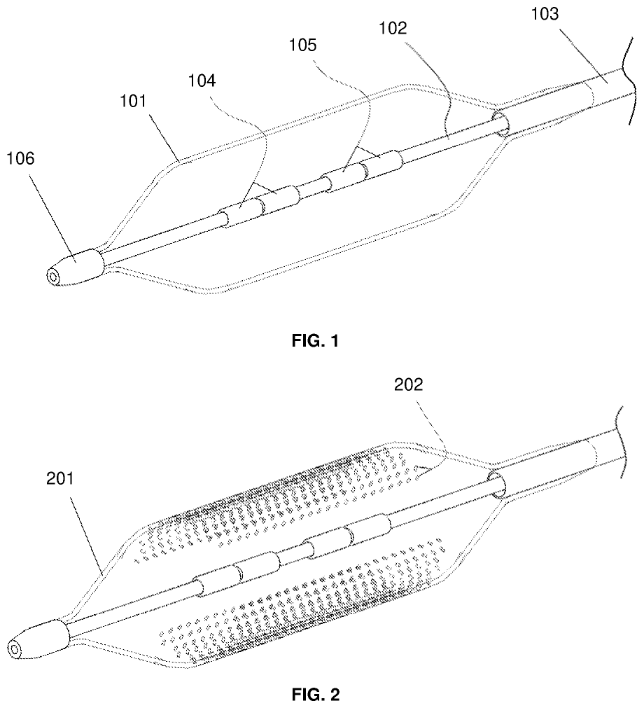

FIG. 1 illustrates a basic shockwave-generating chamber catheter system, comprising a balloon 101, a first elongate member 103, a second elongate member 102, and a plurality of electrode pairs as depicted by 104 and 105, and a nosecone 106. More electrodes pairs can be added as desired. The balloon 101 is connected to both the first elongate member 103 and the second elongate member 102 at their distal ends. This creates a sealed cavity within the balloon such that it is air and water-tight to the outside environment. The balloon 101 is inflatable with a fluid via the annular lumen of the first elongate member 103, and shockwaves may propagate from arc discharges between electrode pairs when a current is passed through the system. A general way to use this catheter would be to inflate the balloon within a section of an atherosclerotic vessel such that the balloon compresses the calcific plaque, then trigger shockwaves that propagate through the fluid of the balloon and disintegrate the calcium.

FIG. 2 shows the first type of drug delivery catheter system making use of a single porous balloon. It is similar to the above configuration in FIG. 1, but with the presence of multiple micro-perforations or micropores 202 on the balloon 201. These micropores may cover the entire surface area of the straight section of the balloon, typically in the medial region. The balloon may be tracked to a stenotic length of vessel and inflated with a drug solution to full size, thereby making contact with the vessel walls. Upon added pressure, the drug will be forced out of the balloon via the micropores at high velocity, aiding in the uptake by the vessel walls. The general diameter of each micropore should lie between about 1-2 μm. During this period of increased pressure, shockwaves may be administered to stimulate the vessel walls into accepting more drug molecules, as well as create microjets that help to accelerate the molecules into the walls.

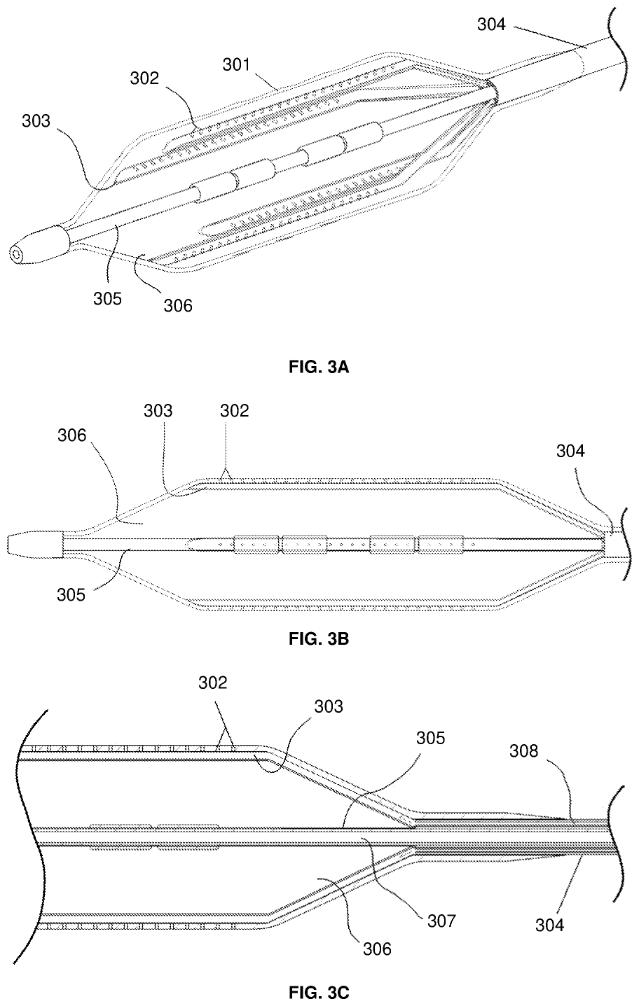

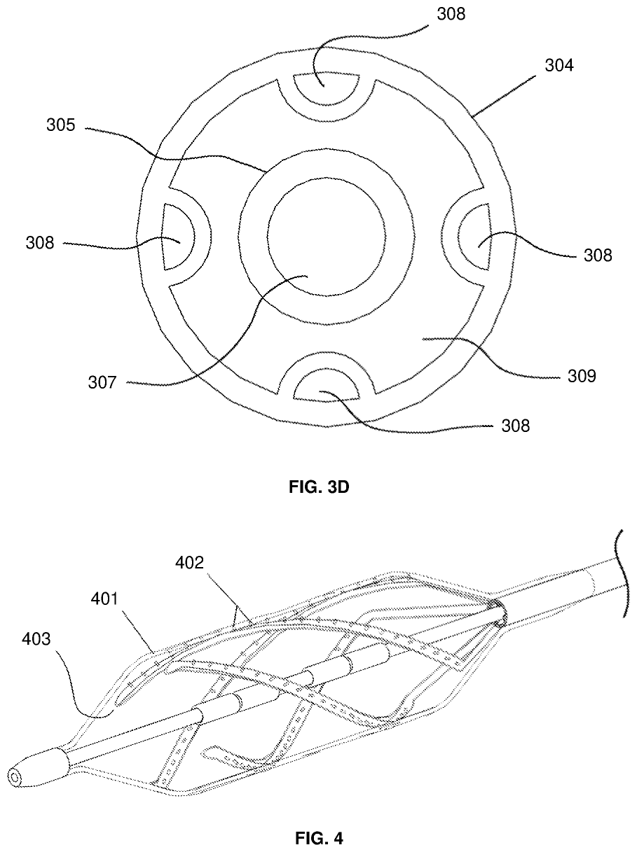

FIG. 3A-D show a second type of drug delivery catheter system, which is a variation of the afore-described single porous balloon, which comprises a number of fluid pockets 303 running along the length of the balloon 301. The micropores 302 only perforate the outer surface of the fluid pockets. 304 is the first elongate member comprising a main fluid channel 309 and multiple smaller fluid channels 308 separately in fluid communication with the balloon chamber 306 and the fluid pockets 303, respectively. 305 is the second elongate member with a wire guide lumen 307. The fluid pockets 303 are sealed off from the balloon chamber 306, thereby preventing flow of liquid or gas between the balloon chamber and the fluid pockets. The balloon chamber 306 is thus separate from the volumes within each fluid pocket 303. The balloon chamber 306 can be inflated with a saline/contrast mixture via the afore-mentioned main fluid channel 309 to compress against a vessel, while the fluid pockets 303 can subsequently be filled with a drug solution via the smaller fluid channels 308 of the first axially extending elongate member. Added pressure such as by forcing more drug into the fluid pockets, or more fluid mixture into the main balloon 301, would cause the drug to exit the fluid pockets via the micropores at high velocity. Shockwaves may then be applied to enhance the drug uptake by the vessel walls. This configuration physically separates the drug from the electrical current, and reduces the volume of drug solution administered per procedure. While the figures show four fluid pockets 303 existing within the balloon, it should be understood that the number of fluid pockets may be increased or decreased according to the amount of coverage desired. For example, the catheters disclosed herein may comprise from 1 to 8 fluid pockets, such as from 3 to 6.

The fluid pockets shown in the figures are straight, but it is also possible to use other patterns and shapes of fluid pockets in order to increase the amount of coverage, while keeping the number of fluid pockets relatively low. FIG. 4 shows one such example of this variation employing the use of a helical pattern, where the four fluid pockets 403 run across at least some part of the circumference of the balloon 401, such that more of the vessel may be exposed to the drug. The micropores 402 also run along the path of each fluid pocket.

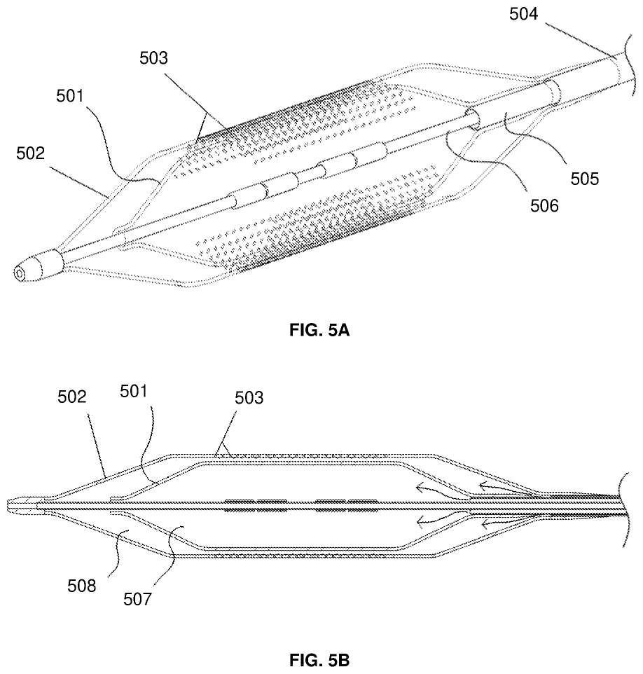

FIG. 5A demonstrates a third type of drug delivery catheter system that utilizes a double-balloon configuration. A second, inner balloon 501 is encapsulated by the first main balloon 502 similar to that in FIG. 1. The second balloon 501 is joined to the second elongate member 506 at a distal region less distal than the attachment of the first balloon 502 to the second elongate member 506. The second balloon 501 is also attached to a new, third elongate member 505. The first balloon is attached to the first 504 and second 506 elongate members. The first balloon 502 comprises perforations 503 similar to those in FIG. 2. The second balloon 501 does not contain perforations. FIG. 5B shows the separation of the two chambers of the balloons 501 and 502, where the second balloon chamber 507 is inflatable with the normal saline/contrast mixture via a fluid channel within the lumen of the third elongate member 505, and the first balloon chamber 508 is inflatable with the drug solution via a fluid channel within the lumen of the first elongate member 504. The second balloon 501 may be filled initially to make first contact with the plaque, followed subsequently by the filling of the first balloon 502 with the drug. Once there is added pressure such as by forcing in more drug into the first balloon chamber 508 or more fluid mixture into the second balloon chamber 507, the drug will be forced out of the micropores at high velocity. Once again, shockwaves will be used to enhance the uptake of drugs during the administration of increased pressure.

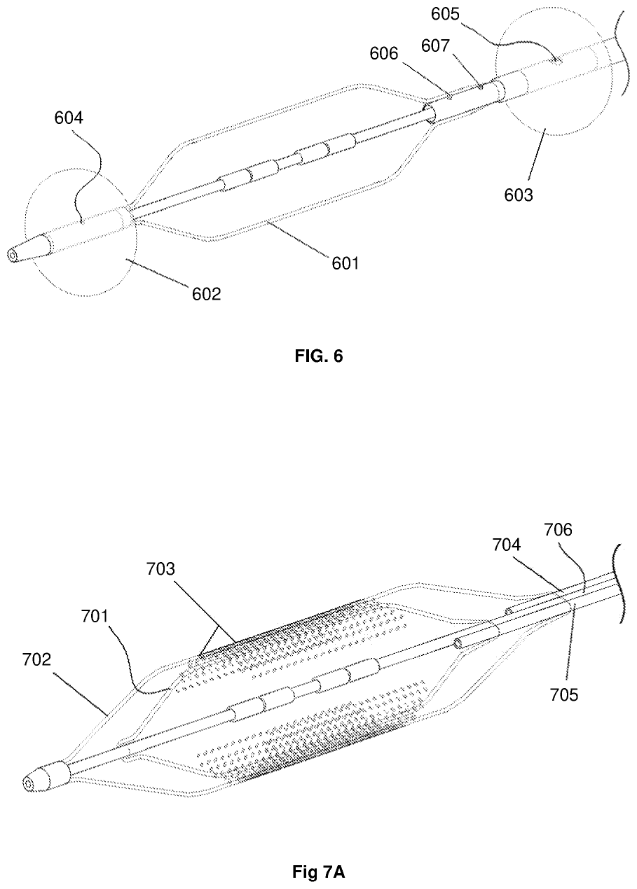

FIG. 6 shows a fourth type of drug delivery catheter system that utilizes an occluding balloon mechanism. The main balloon 601 is again similar to that in the basic system shown in FIG. 1. It is joined by two secondary balloons, one distal 602, and one proximal 603. These balloons, once inflated, will serve to occlude the vessel which the catheter is in. Upon occlusion, blood flow is stopped, and may be evacuated through the opening 607 located either distal or proximal to the main balloon 601. The resulting empty space may be filled with saline via opening 606, either distal or proximal to the main balloon 601. This will aid in flushing blood. The main balloon 601 may then be inflated to a diameter just slightly lower than that of the vessel inner diameter, so no contact is made with the vessel walls. Drugs may then be infused into the space via either opening 606 or 607, and pressurized in order to force the drug into the vessel walls. Shockwaves may then be administered at this point in order to stimulate and enhance the uptake of the drug. The distal and proximal balloons may be inflatable with saline/contrast mixtures via their own ports 604 and 605. In this embodiment, the lumen of the elongate member having openings 606 and 607 (e.g. the first elongate member) may comprise multiple fluid channels, with the openings 606 and 607 connected to different fluid channels. This configuration enables the efficient evacuation of blood through opening 607 and release of saline through opening 606.

The drug delivery catheters of the present invention are based on the concept that the catheter may be navigated to the desired target site (e.g. a specific region in a blood vessel), and then an active agent may be delivered to the target site through a fluid channel within the lumen of one or more of the elongate members (e.g. the first elongate member). Once the active agent has reached the target site, it can be released, for example through micropores in the first balloon or through an opening in one of the elongate members. Shockwaves can then be produced to stimulate uptake of the drugs by the surrounding tissue. This configuration results in a number of advantages. By avoiding the need to coat the exterior of the balloon with active agent, the configuration prevents the washing off of active agent from the exterior of the balloon during navigation to the target site. This reduces side effects associated with drug delivery to non-target sites, reduces the total amount of drug administered and enables a more accurate determination of the actual dose delivered to the target site. The configuration also avoids the need for a bulky protective sheath over the balloons, enabling the catheter to reach narrow vessels.

In all of the above embodiments, the lumen of any or all of the elongate members can comprise multiple fluid channels, with each fluid channel running along the length of the elongate member. This configuration allows the flow of separate fluids to different destinations (e.g. to different balloons, fluid pockets or the exterior of the catheter).

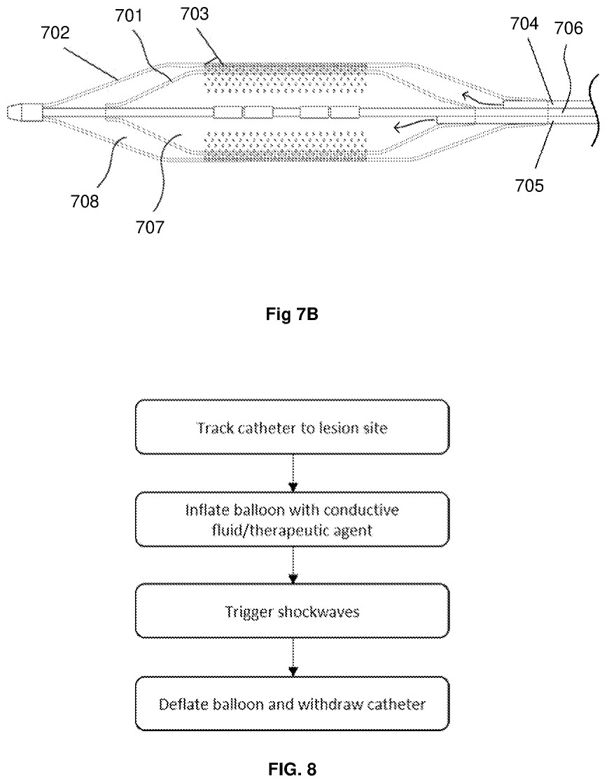

FIG. 7A demonstrates an alternative type of double-balloon configuration similar to that shown in FIG. 5A, where the elongate members are adjacent to each. A second, inner balloon 701 is encapsulated by the first main balloon 702. The second balloon 701 is joined to the second elongate member 706 at a distal region less distal than the attachment of the first balloon 702 to the second elongate member 706. The second balloon 701 is also attached to a new, third elongate member 705. The first balloon is attached to the first 704 and second 706 elongate members. The first balloon 702 comprises perforations 703 similar to those in FIG. 2. The second balloon 701 does not contain perforations. FIG. 7B shows the separation of the two chambers of the balloons 701 and 702, where the second balloon chamber 707 is inflatable with the normal saline/contrast mixture via a fluid channel within the lumen of the third elongate member 705, and the first balloon chamber 708 is inflatable with the drug solution via a fluid channel within the lumen of the first elongate member 704. The second balloon 701 may be filled initially to make first contact with the plaque, followed subsequently by the filling of the first balloon 702 with the drug. Once there is added pressure such as by forcing in more drug into the first balloon chamber 708 or more fluid mixture into the second balloon chamber 707, the drug will be forced out of the micropores at high velocity. Once again, shockwaves will be used to enhance the uptake of drugs during the administration of increased pressure.

FIG. 8-11 are flowcharts describing the general steps that an operator may take in using each type of device. FIG. 8 relates to the use of both a basic shockwave angioplasty catheter like that shown in FIG. 1, and a single microporous balloon drug delivery catheter like that shown in FIG. 2. Both devices comprise just a single balloon with shockwave-emitting electrodes within the balloon chamber. The catheter may be tracked through a bodily vessel via known conventional techniques to the target lesion site. For the basic shockwave angioplasty catheter, the balloon may be inflated with a fluid until the balloon makes full contact with the lesion. Triggering the shockwaves will then cause the calculi within the lesion to crack, thus disintegrating the lesion and increasing the size of the lumen within the vessel. For the single microporous balloon drug delivery catheter, the balloon may instead be inflated with a therapeutic agent to the point that the agent seeps from the balloon into the vessel. Microbubbles may also be introduced into the therapeutic agent mixture. Triggering shockwaves will then stimulate the uptake of the therapeutic agent by the vessel walls. The balloon may then be deflated, and the catheter withdrawn from the body.

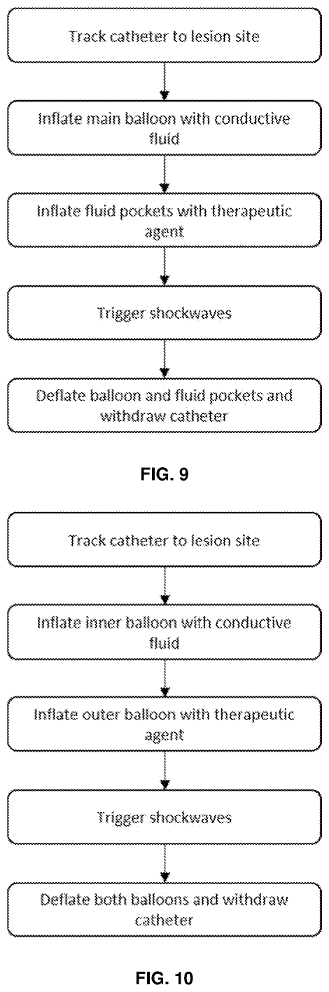

FIG. 9 relates to the use of a single-balloon drug delivery catheter with fluid pockets like that shown in FIG. 3A-C and FIG. 4. The fluid pockets serve to separate the drug solution from the fluid in the main balloon, as well as shield it from the electrical arc generated by the electrodes. After tracking the catheter to the lesion site, the main balloon will be inflated with a fluid until the balloon makes full contact with the lesion. The fluid pockets are then filled with drug solution to the point where the agent seeps from the micropores located over their outer surfaces on the balloon. Triggering shockwaves will then stimulate the uptake of the therapeutic agent by the vessel walls. Microbubbles may also be introduced into the drug mixture, such that their collapse upon impact with shockwaves creates high velocity cavitation jets that enhance the penetrating power of the drug molecules through the vessel walls. The main balloon and the fluid pockets may then be deflated, and the catheter withdrawn from the body.

FIG. 10 relates to the use of a double-balloon drug delivery catheter system like that shown in FIG. 5A-B. Much like the single-balloon with fluid pockets, this system strives to separate the drug solution from both the fluid and electrical arc generation from the inner balloon. At the lesion site, the inner balloon may first be inflated with the fluid, and thereafter the drug solution will be used to inflate the outer balloon, to the point where the agent seeps from the micropores located over the outer surface of the outer balloon. Triggering shockwaves will then stimulate the uptake of the drug by the vessel walls. Microbubbles may also be introduced into the drug mixture, such that their collapse upon impact with shockwaves creates high velocity cavitation jets that enhance the penetrating power of the drug molecules through the vessel walls. The inner and outer balloons may then be deflated, and the catheter withdrawn from the body.

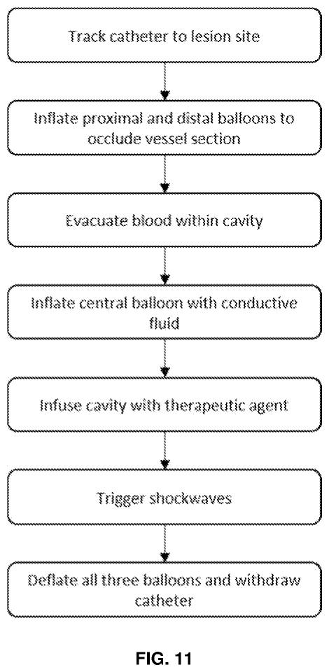

FIG. 11 relates to the use of the multiple-balloon setup as shown in FIG. 6. This system isolates a section of the vessel to be treated in order for more control over the flow and dosage of drug administered. The catheter is tracked to the lesion site and the distal and proximal balloons are positioned respectively, distal and proximal to it. The main central balloon will thus be positioned directly within the lesion. The distal and proximal balloons may then be inflated with contrast in order to occlude the vessel and halt blood flow through this section. The remaining blood may then be evacuated through a port in the catheter and flushed multiple times through the infusion and evacuation of saline. Thereafter, the main central balloon is inflated with a fluid, filling the majority of the volume within the enclosed space, but never touching the vessel walls. Drugs may then be infused through the catheter to fill the cavity under pressure, resulting in maximum exposure of the vessel walls to the agent. Triggering shockwaves will then stimulate the uptake of the therapeutic agent by the vessel walls. Microbubbles may also be introduced into the drug mixture, such that their collapse upon impact with shockwaves creates high velocity cavitation jets that enhance the penetrating power of the drug molecules through the vessel walls. All three balloons may then be deflated to allow blood flow to resume, and the catheter withdrawn from the body.

### EXAMPLES

### Example 1: Temperature Measurements of Shockwave Catheter

In order to confirm that electrical arc discharge does not produce excessive amounts of heat and will not result in a heat injury to a patient, the temperature of shockwave catheters according to the invention was measured.

Shockwave catheters of the type disclosed in FIG. 1, comprising 4 to 24 electrodes arranged in pairs and varying balloon sizes were tested.

Methods

Temperature measurement of the fluid within the balloon.
Elongate members comprising different numbers of electrodes were submerged in a conductive fluid with volume similar to that of the relative balloon.
A thermocouple was inserted into the fluid.
The fluid was warmed to 37±2° C. to mimic the in vivo condition.
Electric arc discharge was generated at a frequency of 1 Hz with voltage range between 1200V-2400V, depending on the number of electrodes. The maximum number of pulse therapy was delivered.
The temperature of the conductive fluid was recorded throughout the pulsing process.

Temperature measurement of balloon surface.
The shockwave balloon catheters were inflated with conductive fluid and submerged under a water bath of 37±2° C.
A thermocouple was fixed on to the balloon surface adjacent to the electrodes.
Electric arc discharge was generated at a frequency of 1 Hz with voltage range between 1200V-2400V, depending on the number of electrodes. The maximum number of pulse therapy was delivered.
The temperature of the balloon surface was recorded throughout the pulsing process.

Results

A maximum of 3° C. increase in temperature of the fluid within the balloon was recorded. A maximum of 2° C. increase in temperature of the balloon surface was recorded.

### CONCLUSION

All recorded temperatures were no higher than 40° C. As such, the risk of thermal injury from the shockwave catheter of the invention is low.

1. A drug delivery catheter comprising:
a first axially extending elongate member comprising a lumen, an outer surface, a distal end and a proximal end;
a second axially extending elongate member comprising an outer surface, a distal end and a proximal end;
a main chamber wall having an outer and inner surface, a distal end, and a proximal end, the inner surface of the main chamber wall defining a main chamber, where the distal end of the main chamber wall is joined to the distal end of the second axially extending elongate member and the proximal end of the main chamber wall is joined to the distal end of the first axially extending elongate member such that the main chamber is disposed over a distal portion of at least the second elongate member; and
one or more shockwave-generating elements disposed within the main chamber, wherein:
the main chamber is in fluid communication with the lumen of the first elongate member;
the drug delivery catheter is configured to deliver an active agent to a target site through the lumen of the first elongate member; and
the one or more shockwave-generating elements comprise two or more electrodes disposed on the outer surface of the second elongate member, the electrodes being configured to enable the arcing of an electrical discharge between electrodes when a potential difference is applied.

2. The drug delivery catheter of claim 1, further comprising one or more chamber walls, each defining a further chamber, wherein each of the further chamber(s) is/are fluidly connected to a lumen of an elongate member and/or to a fluid channel in a lumen of an elongate member, such that each of the further chamber(s) is/are fluidly isolated from the main chamber.

3. The drug delivery catheter of claim 2, wherein a first of the one or more further chambers is disposed over a portion of the second elongate member within the main chamber and the one or more shockwave-generating elements are disposed within the first of the one or more further chambers.

4. The drug delivery catheter of claim 2, further comprising a third axially extending elongate member having an outer surface, a distal end, a proximal end and a lumen, wherein the first of the one or more further chambers is in fluid communication with the lumen of the third elongate member.

5. The drug delivery catheter of claim 4, wherein:
the third axially extending elongate member is partly housed within the lumen of the first elongate member, and extends from the distal end of the first elongate member; and
the second axially extending elongate member is partly housed within the lumen of the third elongate member, and extends from the distal end of the third elongate member.

6. The drug delivery catheter of claim 4, wherein the proximal end of the first of the one or more further chambers is attached to the distal end of the third elongate member, and the distal end of the first of the one or more further chambers is attached to the second elongate member.

7. The drug delivery catheter of claim 1, wherein the two or more electrodes comprise one or more electrode pairs disposed on the outer surface of the second elongate member, the electrode pairs being configured to enable the arcing of an electrical discharge between electrodes when a potential difference is applied, thereby giving rise to shockwaves.

8. The drug delivery catheter of claim 1, wherein the wall of the main chamber comprises a plurality of micropores extending through the wall to fluidly connect the interior of the main chamber to the exterior of the main chamber.

9. The drug delivery catheter of claim 2, wherein:
the one or more further chambers comprise one or more fluid pockets having a distal end, a proximal end, and an outer surface, each fluid pocket running along the length of the interior surface of the main chamber wall or along at least part of the length of the exterior surface of the main chamber wall;
each fluid pocket is enclosed at its distal end; and
the proximal end of the fluid pocket is in fluid communication with a lumen of an elongate member and/or to a fluid channel in a lumen of an elongate member.

10. The drug delivery catheter of claim 9, wherein the one or more fluid pockets are a plurality of fluid pockets.

11. The drug delivery catheter of claim 10, wherein each fluid pocket is in fluid communication with a fluid channel within the lumen of the first elongate member, provided that each fluid channel is fluidly isolated from the main chamber.

12. The drug delivery catheter of claim 9, wherein when a fluid pocket is arranged to run along at least part of the length of the exterior surface of the main chamber wall, the outer surface of each fluid pocket comprises a plurality of micropores extending through the surface of the fluid pocket to fluidly connect the inner volume of fluid pocket to the exterior of the main chamber.

13. The drug delivery catheter of claim 9, wherein when a fluid pocket is arranged to run along the interior surface of the main chamber wall, the wall of the main chamber comprises a plurality of micropores extending through the wall to fluidly connect the interior of the fluid pocket to the exterior of the main chamber.

14. The drug delivery catheter of claim 1, further comprising:
a proximal occluding element disposed over a region of the outer surface of an elongate member comprising a lumen, where said proximal occluding element is more proximal than the main chamber; and
a distal occluding element disposed over a region of the outer surface of an elongate member comprising a lumen, where said distal occluding element is more distal than the main chamber,
wherein the proximal and distal occluding elements are each configured to reversibly occlude a vessel, thereby isolating a portion of said vessel between the proximal and distal occluding elements.

15. The drug delivery catheter of claim 14, wherein:
the proximal occluding element has an inner surface defining an inflatable proximal occluding chamber; and
the distal occluding element has an inner surface defining an inflatable distal occluding chamber.

16. The drug delivery catheter of claim 15, wherein:
the proximal occluding element is inflatable via an opening on the region of the outer surface of the elongate member encompassed by the proximal occluding element, the opening connecting the proximal occluding chamber to the lumen of the elongate member encompassed by the proximal occluding element and/or to a fluid channel within the lumen of the elongate member encompassed by the proximal occluding element; and
the distal occluding element is inflatable via an opening on the region of the outer surface of the elongate member encompassed by the distal occluding element, the opening connecting the distal occluding chamber to the lumen of the elongate member encompassed by the distal occluding element and/or to a fluid channel within the lumen of the elongate member encompassed by the distal occluding element.

17. The drug delivery catheter of claim 16, wherein the proximal occluding element is disposed over the outer surface of the first elongate member proximal to the main chamber;
the distal occluding element is disposed over the outer surface of a distal portion of the second elongate member; and
the second elongate member has a lumen.

18. The drug delivery catheter of claim 14, further comprising an outer axially extending elongate member having a distal end, a proximal end, an outer surface and a lumen, where the first axially extending elongate member is partly housed within the lumen of the outer elongate member and extends from the distal end of the outer elongate member; and
the proximal occluding element is disposed on the outer surface of the outer elongate member.

19. The drug delivery catheter of claim 14, wherein an elongate member comprises one or more openings along its length, the one or more openings being positioned between the proximal and distal occluding elements, each opening connecting a fluidly separate fluid channel within the lumen of said elongate member to the exterior of the catheter.

20. The drug delivery catheter of claim 14, wherein the proximal and distal occluding elements are balloons.
